(19) United States
(12) Patent Application Publication
(10) Pub. No.: US 2016/0367790 A1
(43) Pub. Date: Dec. 22, 2016

(54) DRUG RELEASE PRODUCTS AND METHODS

(71) Applicant: Andrey Belenky, Staten Island, NY (US)

(72) Inventor: Andrey Belenky, Staten Island, NY (US)

(21) Appl. No.: 14/744,319

(22) Filed: Jun. 19, 2015

(51) Int. Cl. A61M 31/00; A61B 5/00; A61B 5/07; A61K 9/00

(52) U.S. Cl. CPC A61M 31/002; A61K 9/0097; A61B 5/4839; A61B 5/073; A61M 2205/50; A61M 2230/005; A61M 2205/82; A61M 2205/3507

(57) ABSTRACT

A drug release products and methods are disclosed. The drug release product has one or more compartments containing drugs and a space with control elements. The control elements control the timing and the dosage of the drug release by opening compartments at particular times. The control elements use information from on-board sensors, from a probe located in another part of the patient's body or from external device to determine the timing of opening the compartments. The product with the unopened compartments and control elements leaves the patient's body through the normal digestive process.

### FIELD OF INVENTION

This invention relates to drug release products and methods.

### BACKGROUND

Drug time release technologies are used in pill tablets and capsules. These technologies use chemical or physical characteristics of the materials comprising a pill or capsule. While these technologies have become sophisticated, their major limitation is the invariable nature of the release. In other words, once a patient swallows the pill or capsule, the drug contained inside is released according to the design without the possibility of altering the release. The timing and the dosage of such release may not be optimal for the particular patient's condition. Accordingly, there is a need in the art for products and methods that would be capable of controlling the timing and the dosage of the drug release based on the information not necessarily available in advance, such as characteristics then existing in the patient's body.

### SUMMARY

The problem that exists in the art is a predetermined nature of the drug release, even for sophisticated drug release technologies presently available in the art. These prior art time release technologies do not take into account information about the patient or the condition to be treated by the drug. In this disclosure, the term “drug” has the broadest possible meaning and includes, but is not limited to, “drug” as defined by the Federal Food, Drug, and Cosmetic Act, (2) “food” as defined by the Federal Food, Drug, and Cosmetic Act, or (3) “dietary supplement” as defined by the Federal Food, Drug, and Cosmetic Act, all at the time of filing of the present disclosure.

The problem is solved by products and methods described in this disclosure, specifically, a drug-containing product, which is preferably a capsule, and associated methods. The capsule has walls (also referred to as “partitions”; in this disclosure, the terms “wall” and “partition” mean the same thing and are used interchangeably.) that form a control space that contains control components and one or more compartments, each containing a drug, which may be the same or different. The control space contains a microprocessor (also referred to as a “processor”; in this disclosure the terms “microprocessor” and “processor” mean the same thing and are used interchangeably), power source, and input/output component. In some embodiments, the control space may include separate memory in addition to the memory built into the microprocessor. In some embodiments, input/output component is connected to one or more sensors. In some embodiments, input/output component is connected to a receiver and optionally to transmitter. The control space and components stored in it, as well as other permanent structures of the capsule, leave the patient's body through the normal digestive process.

The capsule is capable of opening each of the drug compartments and releasing the drug contained in the compartment. The microprocessor provides instructions for opening drug compartments based on certain information about one or more characteristics from on-board sensors or from a probe implanted into the patient's body or located outside the patient's body. The microprocessor may also receive information that it would take into account in determining the timing of different drug compartment opening from an external computer device. After analyzing the information, the microprocessor inside the capsule makes a decision regarding the timing and dosage of the drug release. In some embodiments, the capsule can be completely controlled from the outside by the external device that can override or partially override the behavior of the capsule. Secure wireless protocols that minimize the risk of unauthorized access to capsules' memories are contemplated to

Various embodiments for opening compartments are contemplated. In one embodiment, an outside wall of a compartment is made of a transient material (material that degrades under an influence of one or more stimuli, such as light, heat, electricity, etc.) and the processor exposes the wall to the stimuli, thereby causing the degradation of the wall and release of the drugs stored in the respective compartment. In another embodiment, the outside wall is made of a smart material, such as shape-memory polymer or shape-memory alloy. These materials return to their original shape upon the application of a stimuli. The original shape of the wall may be a book-folded or accordion folded. The modified shape is the shape that completely covers the respective compartment and prevents the release of the drug stored inside until a stimuli is applied to the wall and changes its shape to the original, which enables the drug release. In another embodiment the wall is a made of a piezoelectric material. Upon the application of voltage to the wall, the wall changes its shape creating gaps through which the drug stored inside may be released.

In another embodiment, the release of one or more drugs is accomplished by the relative displacement of the capsule parts. In this embodiment the frame that contains drug compartments is covered by a cover. The cover has holes that, at the time when the patient swallows the capsule, are not aligned with the compartments. When the drug is to be released, the cover is rotated so that one or more compartments is aligned with one or more holes, which enables the drugs stored inside these compartments to be released. While the relative rotation of capsule components is an exemplary embodiment, other embodiments that operate due to the mechanical displacement of parts, such as sliding, are contemplated.

The capsule may be programmed in a variety of ways. The initial programming is preferably done by the manufacturer, but subsequent programming and information input may be done by the entities filling the prescription, medical professionals, or even the patient himself. In this disclosure, the term medical professional should be understood broadly and includes, but is not limited to, doctors, physician assistants, nurses, nurse assistants, home attendants, aides, and various technicians. The capsule may take in consideration various genetic and non-genetic information (such as other drugs taken, family history, etc.) about the patient. In addition, the capsule gathers real-time information about monitored characteristics from the on-board sensors, from the probe implanted into the patient's body, or from an external computer device. Based on all this information and the specifics of the released drug, the microprocessor determined which compartments to open and when to open them. This provides the control over the timing and the dosage of the drug not available in prior art products.

In some embodiments, the behavior of the capsule is controlled by a medical professional. The medical professional may override the programming of the capsule based on personal observations or other information. In these embodiments, the medical professional, through an external computer device, communicates the timing and the dosage of the drug to be released.

The capsule may carry multiple drugs, for example, a secondary drug may be used to reduce side-effect of the primary drug. The capsule may carry multiple drugs, which accomplish similar results, and, based on the acquired information, the capsule releases only one of the drugs. Moreover, the capsule, specifically created for a particular patient's condition may carry multiple drugs. Such a capsule may replace numerous pills that the patient has to take every day.

In one aspect, disclosed, is a drug-containing product comprising a first partition forming, at least in part, a drug-containing compartment, and a microprocessor capable of causing the drug-containing compartment to open by changing a property of the first partition. The property of the first partition may be the physical state, shape, or solubility. The property of the first partition may be chanted through an application of one or more stimuli. These stimuli may be electricity, heat, or light. The drug-containing product may further comprise a sensor capable of monitoring a characteristic, the processor being capable of determining the timing of the drug-containing compartment opening based on the monitored characteristic. The processor is located in the control space inside the drug-containing product. The control space also contains a power source for generation of the stimuli. The drug-containing product also comprising a receiver capable of receiving information and the drug-containing product is capable of determining the timing of the drug-containing compartment opening based on the received information.

Because the drug-containing product contains a microprocessor and other electronic component, saving energy is a consideration. Initially, the capsules may be in an inactive state, with most of the electronic components disabled. When the capsule is activated, all the electronic components may be powered to be fully functional. When the capsule is inactive, only those components that are involved in the activation process are active. This conserves energy while the drug-containing product is inactive. The activation may be accomplished in a number of ways. For example, exposing the drug-containing product to the highly acidic environment may activate the capsule. Alternatively removing the capsule from a vacuum seal or form a special container may activate the capsule. Alternatively, sending an activation signal to the capsule may activate it.

Various methods of use of the drug-containing capsule are possible. The basic method is to activate the capsule, measure or acquire information about a monitored characteristic, analyze the information and release the drug based on the results of the analysis. Various delays may be introduced into the above steps as required. Additionally, the steps that follow activation may be repeated a number of times. Moreover, if the drug-containing capsule contains multiple drugs, the steps can be done simultaneously for various drugs stored in different compartments.

### BRIEF DESCRIPTION OF THE DRAWINGS

FIG. 1 shows a block diagram of the capsule.

FIG. 2 shows a patient who swallowed a capsule, a probe implanted into the patient's body and external devices that monitor the capsules behavior, have the capability to affect the capsule, or both.

FIG. 3 shows vacuum packaging of the capsules.

FIG. 4 shows a special container for the capsules.

FIG. 5 shows an exemplary 3-dimensional view of the capsule, in which

FIG. 6 shows a cross-section of the capsule shown in FIG. 5 along plane A.

FIG. 7 shows a cross-section of the capsule shown in FIG. 5 along plane B.

FIG. 8 shows a 3-dimensional view of a single compartment and adjacent control space for the embodiments in the drug release is accomplished by degradation, melting, or changing of shape of one of the walls.

FIG. 9 shows a 3-dimensional view of a single compartment with two electric leads.

FIG. 10 shows a 3-dimensional view of a single compartment with four electric leads.

FIG. 11 shows a 3-dimensional view of a single compartment with an electric conductor traversing a substantial area of the wall intended to be degraded or melted.

FIG. 12 shows a 3-dimensional view of a single compartment equipped with LEDs.

FIG. 13 shows a wall made of piezoelectric material in its original shape.

FIG. 14 shows a wall made of piezoelectric material in its changed shape after the application of voltage.

FIG. 15 shows a cross-sectional view of one of the compartments in which the permanent walls have protective lips.

FIG. 16 shows the single compartment wall made of a shape-memory polymer or alloy in its altered state.

FIG. 17 shows the single compartment wall made of a shape-memory polymer or alloy in its original state or being folded in half along the vertical axis.

FIG. 18 shows the single compartment wall made of a shape-memory polymer or alloy in its original state or being accordion-folded along the vertical axis.

FIG. 19 shows a cross-section of a single compartment with a wall intended to accomplish the drug release being friction-fitted.

FIG. 20 shows a cross-section of a single compartment with a wall intended to accomplish the drug-release attached to the permanent walls by an adhesive.

FIG. 21 shows a cross-section of a single compartment of the embodiment where the release of drugs is accomplished by melting of a wall with additional outside layer.

FIG. 22 shows a cross-section of capsule with compartments having a curved shape.

FIG. 23 shows a cross-section of capsule with concentric compartments.

FIG. 24 shows a 3-dimensional view of the capsule in which the drug release is accomplished by relative mechanical displacement of parts.

FIG. 25 shows a cross-section of the capsule shown in FIG. 24 along plane D.

FIG. 26 shows a cross-section of the capsule shown in FIG. 24 along plane C.

FIG. 27 shows a 3-dimensional view of the frame with walls making up drug compartments and control space

FIG. 28 shows a 3-dimensional view of the covers.

FIG. 29 shows a cross-section of capsule shown in FIG. 24 with the covers are movably attached to the frame by means of grooves and L-shaped protrusions.

FIG. 30 shows a 3-dimensional view of a capsule in which the drug-release is accomplished by relative mechanical displacement of parts with a sealant.

FIG. 31 shows a cross-sectional view of a capsule in which the drug-release is accomplished by relative mechanical displacement of parts with thick permanent walls.

FIG. 32 shows a cross-sectional view of a capsule in which the drug-release is accomplished by relative mechanical displacement of parts with an elastic lips.

FIG. 33 shows apparatus for supplying instruction and information to capsules.

FIG. 34 is a flow chart of one-time drug release method.

FIG. 35 is a flow chart of one-time delayed drug release method.

FIG. 36 is a flow chart of multiple drug release method.

### DETAILED DESCRIPTION

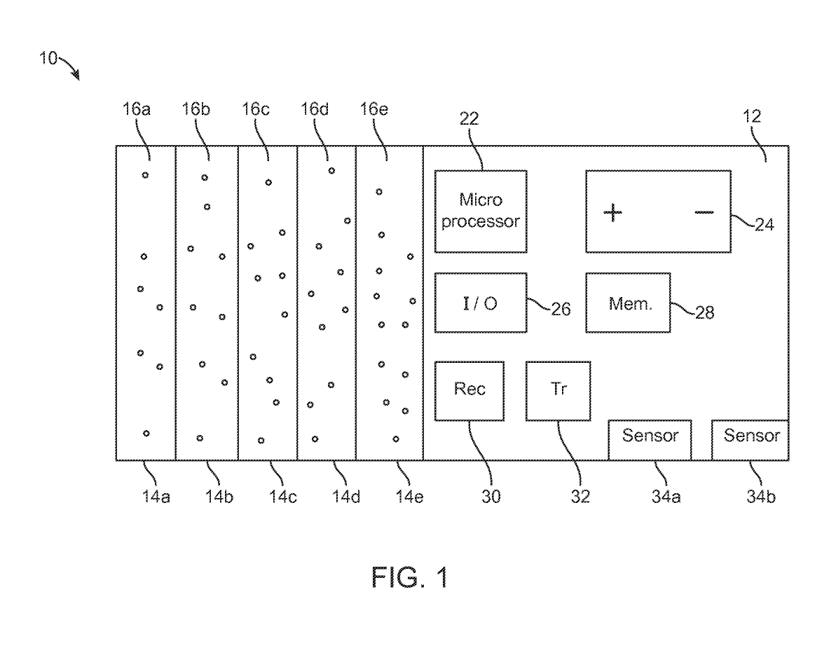

In an exemplary embodiment the drug release product is a capsule. FIG. 1 shows a block diagram of the capsule 10. The capsule has space 12 that contains control components and one or more compartments 14a-14e, each containing drug 16a-16e. Drugs 16a-16e may be the same or different. Drugs 16 may be powder, liquid, gel, solid, gas, nanoparticles, or other forms or a combination thereof. Space 12 contains microprocessor 22, power source 24, and input/output components 26. In some embodiments, space 12 may include separate memory 28 in addition to the memory built into the microprocessor. In some embodiments, input/output components 26 are connected to one or more sensors 34. In some embodiments, input/output components 26 are connected to receiver 30 and optionally to transmitter 32. Capsule 10 also contains a mechanism (not shown in FIG. 1) to separately open each of compartments 14 and release the drug(s) contained in them.

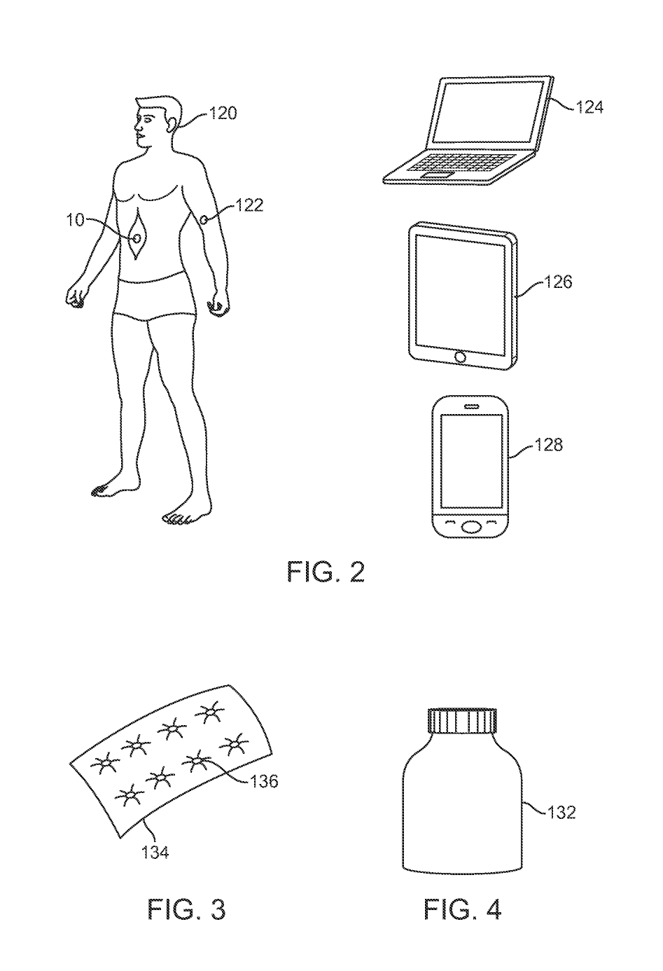

With reference to FIG. 2, after a person (also referred to as a patient) swallows capsule 10, microprocessor 22 receives information about one or more measured characteristics. The receipt of information may be one-time or continuous. Depending on this information, microprocessor 22 causes one or more compartments 14 to open, at the same time or at different times, thus controlling the timing and dosage of drugs 16 release.

When the person swallows capsule 10, sensors 34 measure one or more characteristic, such as the presence or concentration of a particular chemical, temperature, or other parameter, and pass this information to processor 22. Alternatively, receiver 30 receives information from probe 122 implanted into the patient 120 body, but located outside capsule 10, or from outside the patient's body. In some embodiments, probe 122 may be located outside the patient's body and may be a thermometer, blood pressure monitor, oximeter, or the like. Such a probe may have access to additional information through access to blood or other sources of one or more other characteristics. In some embodiments, processor 22 receives information from both onboard sensors 34 and from one or more outside probes 122. Processor 22 analyzes the information and, based on the results of the analysis, instructs one or more compartments 14 to open. Once the target compartments 14 open, drug 16 stored inside compartments 14 is released into the area where capsule 10 is located at the time, and ultimately reaches the blood stream of the person.

In some embodiments, processor 22 analyzes the received information once, but in some other embodiments, processor 22 may analyze the information multiple times, or continuously for a predetermined period of time. The initial analysis may be triggered, for example, by a change in the surrounding of capsule 10. For example, in some embodiments, the detection of gastric acid by sensors 34 may be the trigger. In other embodiments, the trigger may be the removal of capsule 10 from a special container 132 shown in FIG. 4, or the removal of capsule 10 from a vacuum wrapper 134 shown in FIG. 3, where each capsule is stored inside an individual vacuum-sealed space 136. After the capsule is removed from vacuum wrapper 134 it is exposed to air, atmospheric pressure, or another normal condition outside the wrapper that triggers the monitoring by processor 22. In some other embodiments, the trigger may be the reading of one or more monitored characteristics. In some embodiments, capsule 10 may be activated manually, by, for example, pressing on a designated spot of the capsule and thereby completing an electric circuit. In some embodiments, capsule 10 may be activated form external device 124, 126, 128. Once the trigger occurs, processor 22 may begin monitoring one or more characteristics of interest and, according to how it is programmed, releasing drug 16 by opening compartments 14. Such triggering enables capsule 10 to be in a stand-by mode, where only the components responsible for detecting the trigger are active, but the remaining components are inactive and consume no or little power. This allows capsule 10 to be functional for a longer time period.

As the capsule travels through the gastrointestinal tract, one or more compartments 14 may open at various times. The capsule with unopened compartments 14, components in space 12, and other permanent structures exits the body via the normal digestive process.

Turning to FIG. 2 again, in some embodiments the release of drug 16 may be controlled from the outside by a medical professional or by the patient himself. In these embodiments, transmitter 32 or probe 122 may send information regarding one or more monitored characteristic. This information may be transmitted wirelessly to one or more external devices outside the patient's body, such as computer 124, tablet 126, phone 128, or another dedicated device. The device(s) may have installed special software for communicating with capsule 10 and probe 122. Alternatively, the devices may be a piece of equipment dedicated to this task. In some embodiments the external device is programmed to communicate only with specific capsules, or capsules assigned to a specific patient. The computer device outside the patient's body receives the information from capsule 10, probe 122, or both and analyzes the information. The computer device may have more sophisticated software than may be possible to run on microprocessor 22 and it can also have access to information about patient 120. The information may be as simple as the patient 120's weight and age, but may include his complete medical history, family medical history, and genetic information. Based on the available information, the computer device may generate information that it communicates back to processor 22 to optimize the decision regarding the timing and the dosage of drug 16 release. Alternatively, the patient or the medical professional may analyze the information and make a complete or partial decision regarding the time and the dosage of drug 16 release. The decision may be regarding the actual time and dosage of drug 16 release, or may be a more complex conditional instruction, such as, based on the characteristics monitored and transmitted by microprocessor 22. In general, the decision-making logic may be split between microprocessor 22, outside computer system such as computer 124, tablet 126, or the phone 128, and a human such as patient 120 or a qualified medical professional.

Preferably, components inside space 12 are miniaturized. The dimensions of capsule 10 are limited by the ability of a patient to swallow it. Taking the size of a typical pill as a reference, the diameter of space 12 may be as large as several millimeters. Necessary components presently known in the art can fit inside such a space. Advents in nanotechnology produced electronic components such as microprocessors, memory, receivers and transmitters, and other components with the dimensions of less than 1 mm. Also sensors can be implemented as a lab-on-a chip (LOC), microelectronic systems (MEMS), or Micro Total Analysis Systems. Devices that can fit inside space 12 and perform various chemical analyses are available and can be used as part of capsule 10. In general, sensors 34 can perform complete chemical analysis of the surrounding environment within relatively short period of time and provide the necessary information to microprocessor 22. In some embodiments, sensors 34 are configured to measure only one or more characteristics. In some other embodiments, microprocessor 22 may instruct sensors 34 what characteristic to measure and after the measurement is made, it can instruct sensors 34 to measure one or more other characteristics to obtain information required for improving treatment. Some information cannot be obtained from analyzing chemicals in the gastrointestinal system; such information may only be available through, for example, blood testing. If microprocessor 22 relies on the information obtain from blood testing, then microprocessor 22 communicates with an implanted probe 122 shown in FIG. 2. Also external monitoring devices such as thermometer, blood pressure monitor, oximeter, or another device monitoring one or more patient's characteristics from the outside. Alternatively, information about one or more characteristic inaccessible to sensors 34 can be supplied by an outside computer device, such as device 124, 126, or 128.

In some embodiments, power supply 24 is a battery. Power supply 24 must be capable of producing enough energy to activate capsule 10, operate all control components once capsule 10 is activated, and to open all compartments 14. If in a particular embodiment the power supply 24 does not fit into space 12, one or more of compartments 14 may be used to store additional, backup power supplies, or one or more of compartments 14 may be combined with space 12, so space 12 is larger. In some embodiments, power supply 24 may store energy of the body heat. In some embodiments, power supply 24 may store the vibrational energy of heart beating. Any other electronic component that is preferably located in space 12 may be located in one or more dedicated compartments 14.

Various embodiments of capsule 10 are contemplated. These embodiments may vary greatly in the shape and size of capsule 10, the shape and size of compartments 14, the size and location of space 12, the drug release mechanism, and other features.

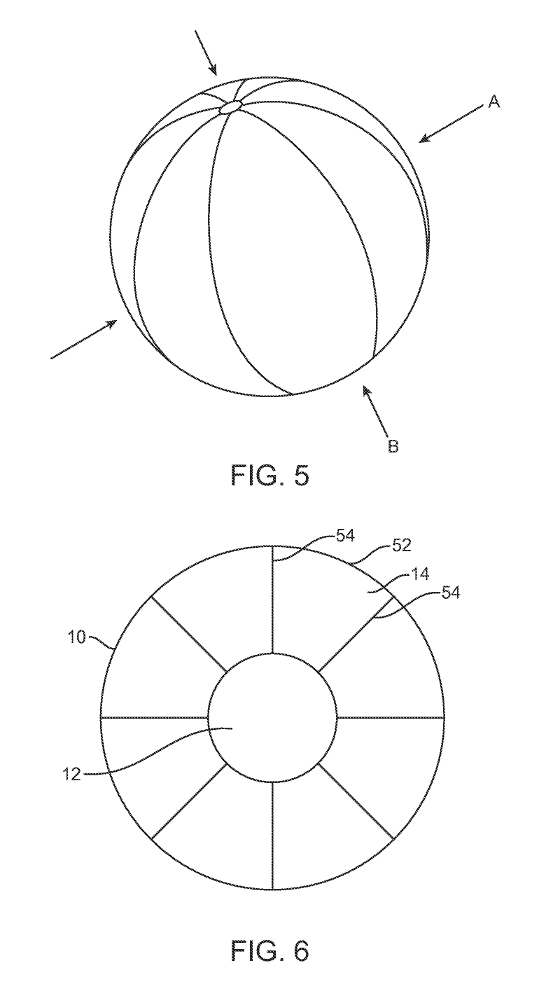

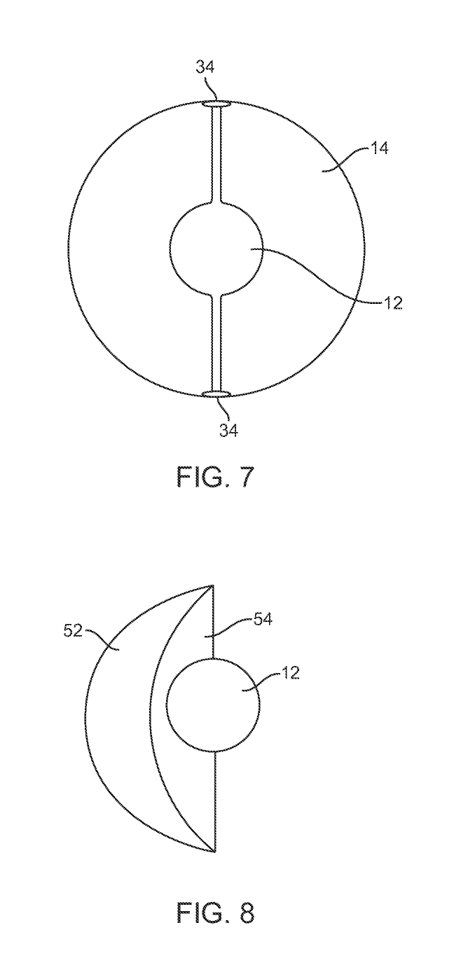

FIGS. 5-8 show a preferred embodiment of capsule 10. In this embodiment, capsule 10 is a sphere with space 12 located in the center, and compartments 14 having the wedge shape. FIG. 5 shows an outside 3D perspective of capsule 10. FIG. 6 shows a cross-section of capsule 10 along plane “A” shown in FIG. 5. FIG. 7 shows a cross-section of capsule 10 along plane “B” shown in FIG. 5. FIG. 8 shows a single compartment 14 together with space 12. In some embodiments, compartments 14 have identical shape and size, while in some other embodiments, the shape, size, or both may be different for different compartments Inner walls 54, separating compartments 14, are made of non-toxic or low-toxicity indigestible material. Cellulose is one such suitable material because it is not toxic, is indigestible by humans and has a relatively high melting point of 230° C. or above. Other indigestible substances such as indigestible forms of polysaccharides, polymers, dextrin, and other materials can be used in construction of walls 54.

Following microprocessor 22 instructions, outer walls 52 change their properties creating openings through which drugs 16 is released. In this embodiment, walls 52 are made of an indigestible transient material that dissolves under the influence of one or more stimuli, such as electricity, heat, light or some other stimulus, or a combination of thereof. The term “transient materials” has been used to describe a class of programmable degradable materials. It is sufficient to accomplish full or partial degradation (dissolution) of walls 52. It would be sufficient to have an aperture in wall 52 for the release of drug 16 stored in the respective compartment 14 to begin.

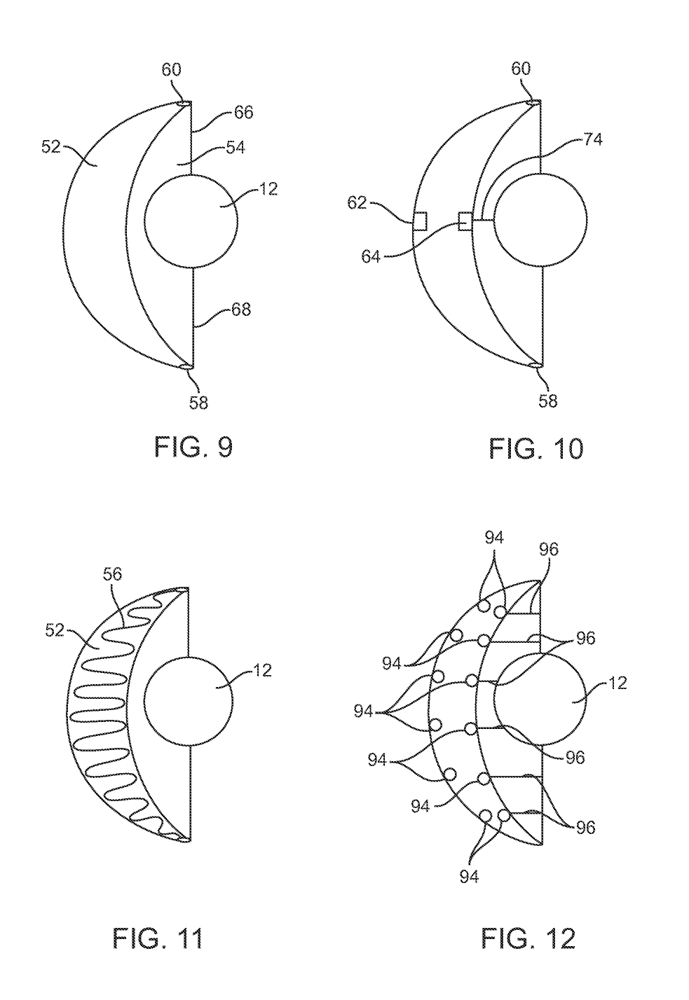

The preferred stimulus for triggering the degradation of wall 52 material is electricity. To accomplish this, at least two electric leads 58 and 60 are in contact with wall 52, as shown in FIG. 9. Schematically, electric leads 58 and 60 are connected, through electronic circuitry, to power source 24 by conductors 68 and 66, respectively. As discussed below, once the decision to open the compartment is made by microprocessor 22 or another device or person, the electronic circuitry applies voltage to leads 58 and 60. The number of electric leads can be increased as needed for a particular application, for example, to decrease the time of degradation or to increase the area of the aperture. Preferably, the electric leads are located within the permanent structures of capsule 10, such as walls 54. In FIG. 9, two electric leads 58 and 60 are in the vicinity of the poles of capsule 10. FIG. 10 shows another embodiment, in which additional electric leads 62 and 64 extend through partition walls 54. As shown in FIG. 10, conductor 72 provides electric current to lead 64. Another conductor (not shown in FIG. 10) provides electric current to lead 62. When, based on the collected data, processor 22 instructs power source 24 to apply voltage to one or more walls 52, the voltage serves as a trigger to degradation of walls 52 to which the voltage is applied. After the degradation is triggered, target walls 52 begin to dissolve. The transient material making up walls 52 is preferably selected with a short degradation time of a several seconds or minutes. In some embodiments, the transient material is selected to have a longer degradation time, to provide for an additional delay. Preferably, the transient material making up walls 52 has low or no toxicity. Also, preferably, the material making up walls 52 is indigestible. This ensures that if the degradation of walls 52 is not triggered, drugs 16 in respective compartments 14 are not released into the patient's body. It should be understood that the number and location of leads and associated conductors for each compartment may vary depending on various parameters including power consumption and the nature of the material making up walls 52.

In an alternative embodiment heat triggers the degradation of the transient material making up walls 52. One way to generate heat is to pass electricity through the material. Depending on the electric resistance of the material, the electricity may be applied at two points of wall 52 as shown in FIG. 9 through leads 58 and 60. Alternatively, if the electrical resistance of the material is high to the point that the material is non-conductive, then an electric conductor may be used to heat up the transient material making up walls 52. FIG. 11 shows conductor 56, which can be thought of as a thin wire snaking adjacent to wall 52 on the inner or the outer surface, or on the inside of wall 52. Conductor 56 covers at least a portion of wall 52 area. In some embodiments, conductor 56 is a layer of conductive material built into wall 52 by a multilayer substrate manufacturing processes. In some embodiments wall 52 with conductor 56 may be manufactured by a spraying process. In some embodiments conductor 56 degrades together with wall 52. In some embodiments conductor 56 does not degrade together with wall 52. Considering a single compartment 14, when electricity is applied to conductor 56 by processor 22 and the power source 24, it generates heat, which causes wall 52 to degrade, which in turn allows the release of drug 16 stored inside compartment 14.

In some embodiments degradation of walls 52 may be triggered by light rather than electricity and temperature. In these embodiments one or more Light Emitting Diodes (LEDs) may be mounted against walls 52, as shown in FIG. 12. When microprocessor 22 decides to open compartment 14, it would light the one or more LEDs 94 inside that compartment, which would trigger the degradation or shape change in the material making up wall 52 of the compartment. LEDs 94 are preferably encapsulated into indigestible clear material that passes light, so that when compartment 14 are opened, LEDs 94 do not contaminate the patient's blood and gastric fluids and do not harm the patient. Preferably, LEDs 94 are connected to power supply 24 in a way that electricity is applied to them once the decision to open a given compartment 14 is made by microprocessor 22, an outside computer device, such as devices 124, 126, 128, or a human operating such a device. LEDs 94 are powered by power supply 24 through conductors 96, which in this embodiment provide both the positive and negative leads.

In some embodiment, compartments 14 open by altering the shape of walls 52. This is accomplished through the use of smart materials, which are defined as materials that have one or more properties that can be significantly changed in a controlled fashion by external stimuli, such as stress, temperature, moisture, pH, electric or magnetic fields. For example, turning to FIG. 8, in some embodiments, walls 52 are made of a piezoelectric material, such as polyvinylidene fluoride (PVDF). PVDF is characterized by the ability to change its shape either by expanding or contracting when voltage is applied to it. Numerous other piezoelectric materials can be used. Preferably the material making up walls 52 is of low or no toxicity and is indigestible.

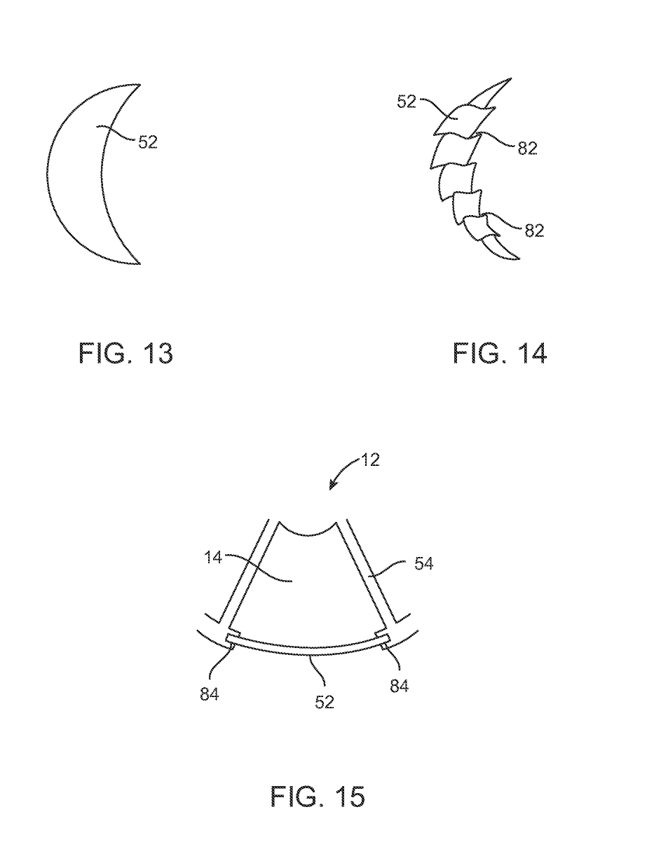

In the initial state, wall 52 covers the entire opening between two adjacent walls 54. FIG. 13 shows wall 52 of one compartment 14 having the original shape. FIG. 14 shows the same wall 52 after electric voltage was applied to it under the control of microprocessor 22. As discussed above, the application of voltage causes wall 52 made of piezoelectric material to change its shape. The change in shape results in multiple openings or gaps 82, through which drug 16 stored inside compartment 14 is released. It should be noted that it may not be possible to determine how wall 52 changes its shape, but practically any change in shape of wall 52 would create one or more openings through which drug 16 is released. Walls 52 are preferably attached to the permanent structures of capsule 10, for example walls 54. In some embodiments, walls 52 are attached to walls 54 close to one of the poles of capsule 10. This provides larger openings 82 than if wall 52 are attached to walls 54 in two or more places.

Some embodiments of capsule 10 have structures preventing walls 52 form exposing sharp edges that form when walls 52 change the shape, which may potentially damage internal organs. For example, in the embodiment shown in FIG. 15, which shows a cross-section of one compartment 14, walls 54 have lips 64 that prevent walls 52 potentially sharp edges to protrude beyond capsule 10 after walls 52 change shape and hide those sharp edges. This reduces the risk of damage to the internal organs caused by the potentially exposed sharp edges.

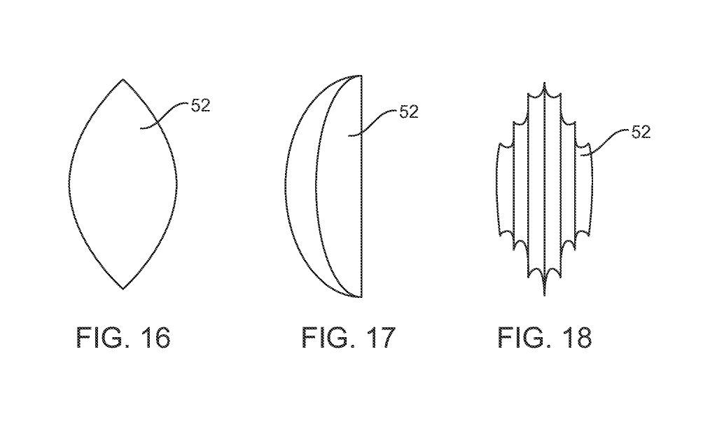

In some embodiments shown in FIGS. 8, 16-18, walls 52 are made of a shape-memory polymer or shape-memory alloy. These materials have the ability to return to the original state after they have been deformed. The trigger for the change may be heat, electricity, light, or another stimulus, or a combination of stimuli. Referring to FIG. 8 that show the general structure of a single compartment, in this embodiment, wall 52 is made of a shape-memory polymer or shape-memory alloy. FIG. 16 shows the altered state of wall 52 that preferably fully closes compartment 14 and prevents the release of drug 16 stored inside compartment 14. FIGS. 17 and 18 show different embodiments of the possible original shape of wall 52, which do not fully close compartment 14 and conversely facilitate the release of drug 16. For example, FIG. 17 shows wall 52 folded in half and FIG. 18 shows accordion-folded wall 52. Upon application of one or more stimuli to the wall 52 in the altered state, shown in FIG. 16, as explained above, wall 52 returns to its original shape (examples of which are shown in FIGS. 17 and 18) thereby opening compartment 14 and releasing drug 16 stored inside.

In connection with the embodiment of wall 52 shown in FIG. 17, a stimuli, such as voltage is applied to wall 52 when it is in the altered shape, causing the change in its shape, which brings wall 52 to its original folded-in-half state. In connection with embodiment of wall 52 shown in FIG. 18, wall 52 undergoes an accordion fold along the axis between the two poles of capsule 10. Other embodiments are contemplated as well. For example, wall 52 may concave inside or undergo another change in shape that would provide an opening for drugs 16 to be released from the respective compartment 14. If the trigger to changing the shape is electricity, the electric leads can be connected to wall 52 or by means of electric conductor 56, as explained above.

In general, electricity, heat, light, or one or more other stimuli, or a combination of stimuli, as discussed above in connection with other embodiments can be used to alter wall 52's shape or properties.

With reference to embodiments where walls 52 are made of piezoelectric materials, shape memory polymers or shape memory alloys, walls 52 may be attached to walls 54 in a variety of different ways. One way of attaching walls 52 is friction fitting. FIG. 19 shows a cross-section of a single compartment 14 and adjacent space 12. In this embodiment, wall 52 is slightly larger than the space formed by adjacent walls 54. Wall 52 is inserted by means of mechanical pressure in the opening. The edges of wall 52 are deformed during manufacture or as a result of the insertion under pressure. The force of the material deformation is what holds wall 52 in place. The force holding wall 52 in place should be less than the force under which wall 52 changes shape when voltage (or other stimuli) is applied to wall 52.

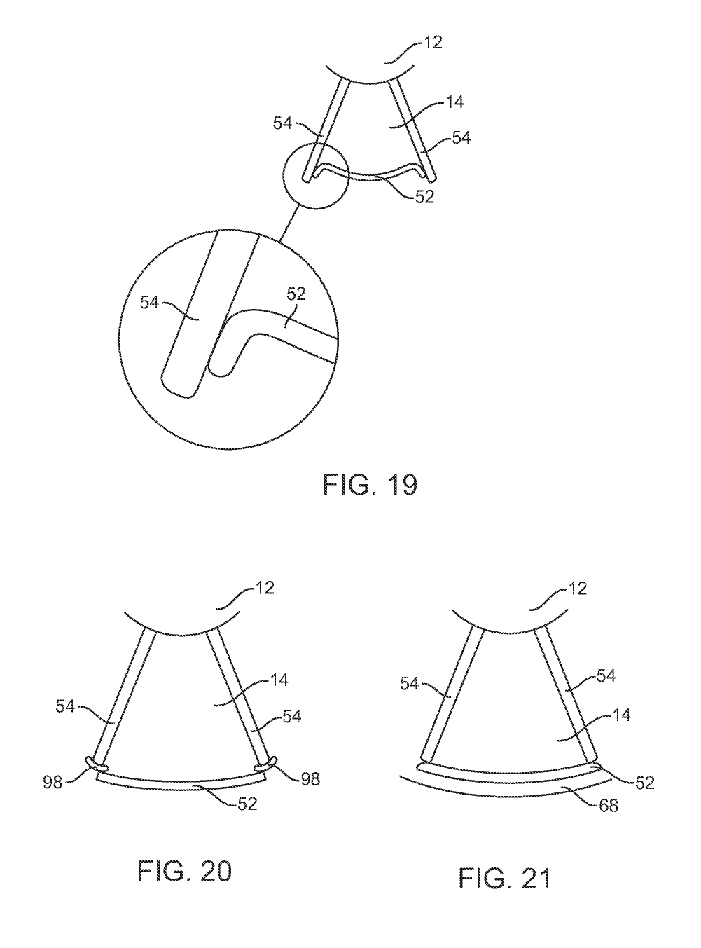

Alternatively, walls 52 are attached to walls 54 by a low or no toxicity indigestible adhesive. FIG. 20 shows a cross-section of a single compartment 14 and adjacent space 12. As shown in FIG. 20, wall 52 is attached to walls 54 by adhesive 98. An examples of such an adhesive is an indigestible form of dextrin, but other preferably low or no toxicity indigestible adhesives are can be used as well. As shown in FIG. 20, the layer of adhesive 98 provides a seal preventing undesirable escape of drugs 16 from compartments 14. In this embodiment, the adhesive force that holds wall 52 in place is less than the force generated by voltage, heat, light (or other stimuli) applied to wall 52 that changes its shape.

In some embodiments, walls 52 melt when heat is applied to them. In these embodiments outer walls 52 of compartments 14 are made of an indigestible, non-toxic or low-toxicity material that has a melting point above normal body temperature (36.6° C.), for example, of above 40° C. and below the temperature of thermal injury of approximately 50° C. Some forms of paraffin or wax have these properties. For example bayberry wax and soy wax are known to have melting points of approximately 45° C. Also, several non-toxic types of paraffin have similar properties. The basic structure of capsule 10 is similar as the one shown in FIGS. 5-8. In this embodiment, the opening of compartments 14 is accomplished by heat that melts wall 52.

Referring again to FIG. 8 that shows one isolated generic compartment 14 and space 12, in this embodiment, wall 52 melts under the influence of heat, which is preferably generated by electric current. Wall 52 is made of the material with poor electric conductivity, such as wax or paraffin, and conductor 56 is built into the wall as shown in FIG. 11. Conductor 56 can be thought of as a thin wire snaking inside wall 54 and covering the majority of its area. In some embodiments, conductor 56 is a layer of conductive material built into wall 52 by a multilayer substrate manufacturing processes. In some embodiments, wall 52 with conductor 56 may be manufacturing by a spraying process. When electricity is applied to conductor 56, it generates heat, which causes parts of wall 52 to melt. Conductor 56 may also melt together with wall 52 or not melt and remain as part of the permanent structure of capsule 10.

In another embodiment, shown in FIG. 21, the principal operation remains the same, however, the capsule has an additional outside layer made of non-toxic digestible material, such as the ones used in prior-art drug capsules, for example animal proteins or plant polysaccharides. In this embodiment, processor 22 opens one or more compartments 14 by generating heat by means of electric current through or along walls 52 before layer 68 is dissolved by gastric acid. This allows for a greater flexibility of wall 52 material characteristic because a material with a higher melting temperature can be used, since any heat and corresponding rise in temperature would be inside the layer 68. This embodiment is particularly suited for application with a single release of drug 16. Because the digestion of the outside layer 68 takes certain time, processor 22 has sufficient time to melt walls 52, which during the melting process are still encapsulated within layer 68. After layer 68 breaks down, some compartments 14 have been opened because the respective walls 52 melted, at least in part.

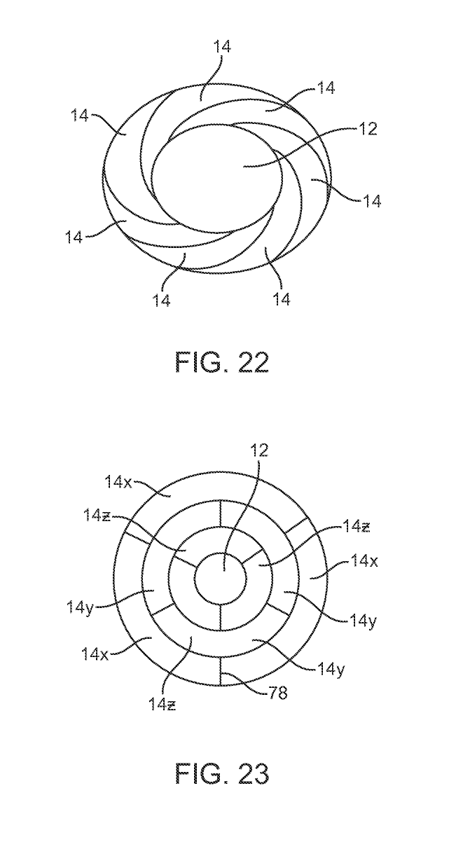

Capsule 10 may take a variety of different shapes. Preferably the shape of capsule 10 is a sphere or ellipsoid, but other shapes may also be used. The shape and dimensions of compartments 14 and space 12 may also be different. In some embodiment the shape of compartment 14 is designed in a way that minimizes sharp edges after one or more of compartments open. This is especially suitable in the embodiments where opening of compartments 14 is accomplished through the degradation of transient materials or melting. FIG. 22 shows a cross-section of an embodiment of capsule 10 with compartments 14 having a curved shape. This shape may minimize the risk of injury if only some of compartments 14 open. FIG. 23 shows a cross-section of another embodiment of capsule 10 where space 12 is at the center of capsule 10 and compartments 14 are concentric around space 12. This embodiment has certain limitations, for example, the drug in the outside compartment 14x must be released first, before other the drugs in other compartments, 14y, and 14z in FIG. 23. In some embodiments compartments 14x all open at the same time. In this embodiments spacer walls 78 are made of the same material as the corresponding walls to be degraded or melted. The advantage of this configuration, is that no matter how many compartments are opened, the capsule remains spherical. In some embodiments, compartments 14 on the same level do not necessarily open all at the same time. In this embodiment, spacer walls 78 may be curved to minimize the presence of sharp edges.

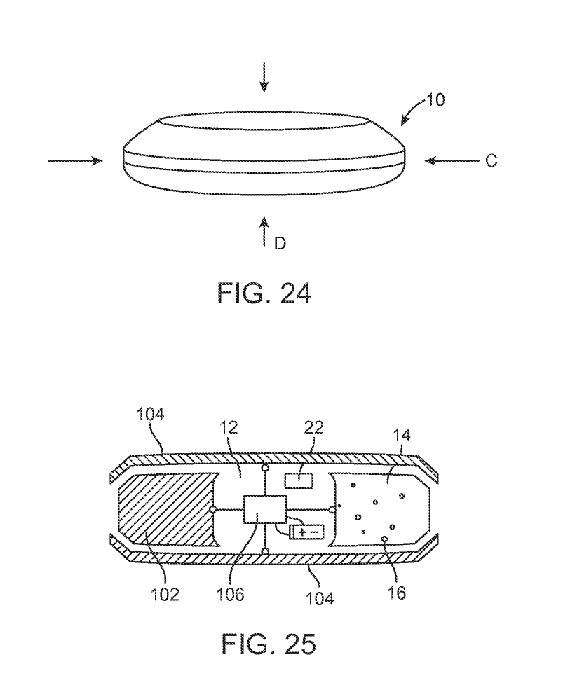

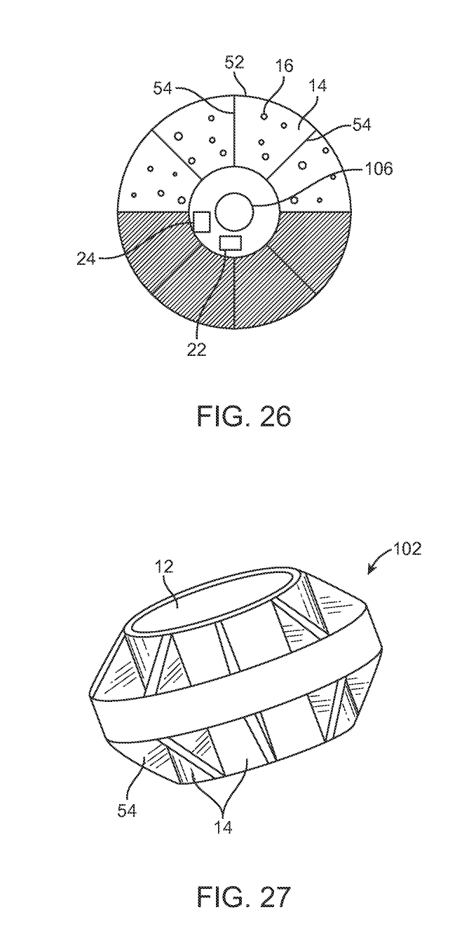

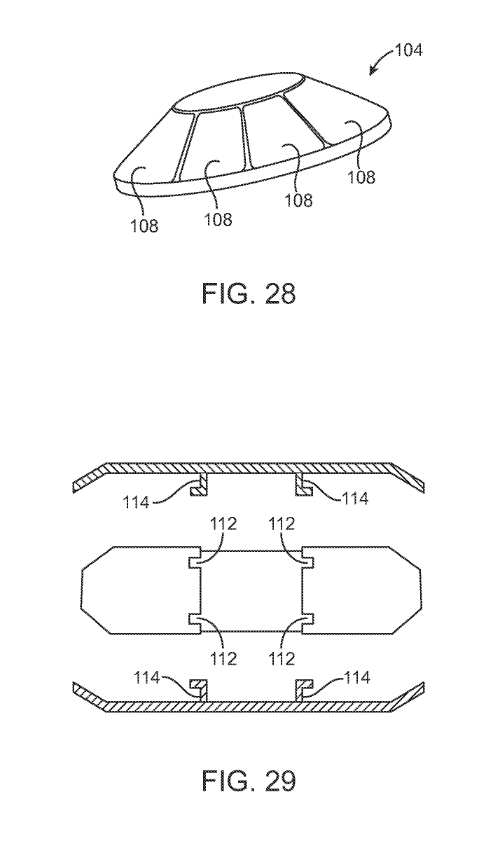

In some embodiments, compartments 14 of capsule 10 may be opened by mechanical force, specifically by mechanical displacement of parts comprising capsule 10. FIG. 24 shows a 3D view of this embodiments of capsule 10. FIG. 25 shows cross-section “C” of capsule 10 shown in FIG. 24. FIG. 26 shows cross-section “D” of capsule 10 shown in FIG. 24. As seen in FIG. 25, in this embodiment capsule 10 comprises frame 102. Frame 102, a 3D view of which is shown in FIG. 27, has walls that form hollow compartments 14, which are filled with drugs 16 and space 12 containing a microprocessor 22, power source 24, and input/output components 26. In some embodiments, space 12 may include separate memory 28 in addition to the memory built into the microprocessor. In some embodiments, input/output components 26 are connected to one or more sensors 34. In some embodiments, input/output components 26 are connected to receiver 30 and optionally to transmitter 32. In this embodiments, space 12 also contains motor 106. Motors that are several millimeters or even one millimeter in all dimensions are known in the art. Preferably the body with the stator of motor 106 is connected to frame 102. The rotor of motor 106 is attached to covers 104 that are movably fitted at the top and bottom of frame 102. FIG. 28 shows a 3D view of cover 104. In this embodiment, frame 102 has four compartments 14 spanning 180°. Similarly, each of covers 104 have four openings 108. When a patient swallows capsule 10, compartments 14 and openings 108 are in complete misalignment. In other words, all compartments 14 are closed. As microprocessor 22 analyzes the received information, it instructs motor 106 to rotate the rotor, with attached covers 104, by a certain number of degrees. For example, the rotation by 45° opens one of the four compartments, the rotation by 90° opens two of the four compartments, etc. As with other embodiments, the release of drug 16 may happen gradually. For example, at one time, the motor can rotate covers 104 by 45° and then at a later time by another 90°. This accomplishes a gradual release of drug 16 stored inside compartments 14. It should be understood that capsule 10 with four compartments 14 of the same size is only one exemplary embodiment. The number and the size of compartments 14 may vary greatly depending on the intended application. Moreover, different drugs 16 may be stored in different compartments 14. In some embodiments, as shown for example in FIG. 31, walls 54 are thick to minimize the risk of accidently releasing of drug 16 due to misalignment of parts.

Turning to the details of the structure of capsule 10 according to this embodiment. FIG. 25 shows cross-section “D” of FIG. 24. Space 12 contains microprocessor 22, power source 24, and input/output component 26. In some embodiments, space 12 may include separate memory 28 in addition to the memory built into the microprocessor. In some embodiments, input/output component 26 are connected to one or more sensors 34. In some embodiments, input/output components 26 are connected to receiver 30 and optionally to transmitter 32. The rotor of motor 106 is attached to covers 104 at the top and bottom. The stator of motor 106 is attached to frame 102. In FIG. 25, to the right of space 12 is one of compartments 14. To the left is unused space of frame 102. It should be noted that in this embodiment a substantial amount of space of frame 102 and capsule 10's volume is unused. This space can be used by an additional power supply, probes, sensors or other permanent components of the capsule.

FIG. 26 shows cross-section “C” in FIG. 24. In FIG. 26 space 12 is located in the middle of frame 102. Inside space 12 are motor 106 microprocessor 22, power source 24, and input/output components 26. In some embodiments, space 12 may include separate memory 28 in addition to the memory built into the microprocessor. In some embodiments, input/output components 26 are connected to one or more sensors 34. In some embodiments, input/output components 26 are connected to receiver 30 and optionally to transmitter 32. FIG. 26 also shows compartment 14 and drugs 16 stored in them.

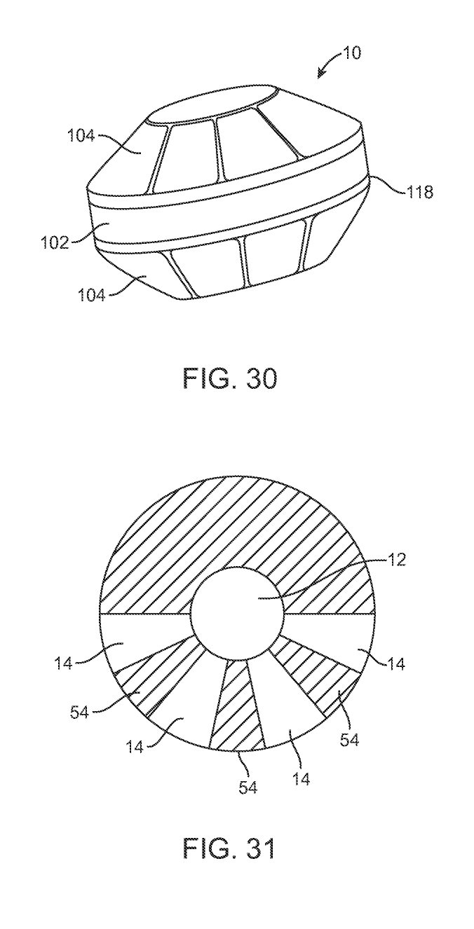

FIG. 29 shows cover 104. In some embodiments, covers 104 are held in place by means of attachment to motor 106. In some embodiments, covers 104 are movably attached to frame 102. For example, as shown in FIG. 29, space 12 has grooves 112 and covers 104 have L-shaped protrusions 114. These L-shaped protrusions snap into grooves 112 and ensure that covers 104 do not separate from frame 102. Other apparatus known in the art for preventing the separation of covers 104 from frame 102 are contemplated, while they are movably attached to each other.

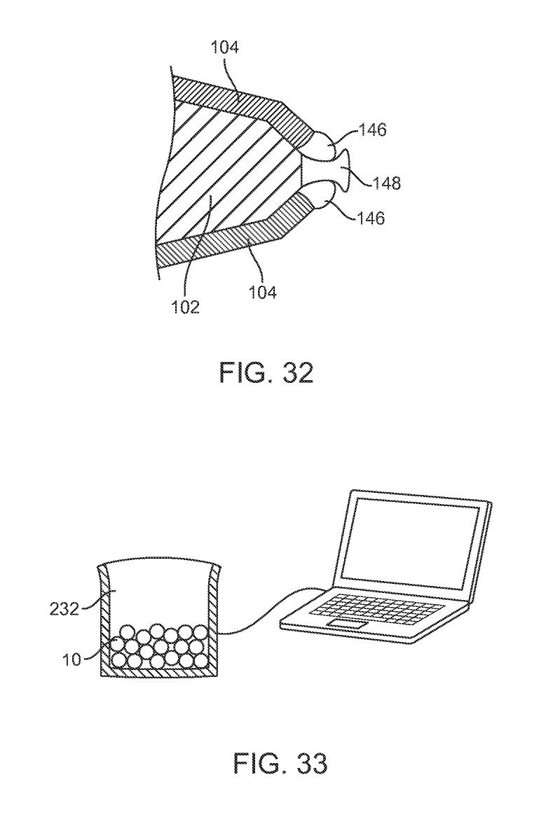

To seal the gap between covers 104 and frame 102, a sealant 118 can be used as shown in FIG. 30. The sealant is preferably non-toxic indigestible viscous material, such as a form of natural latex or synthetic rubber. Also, sealant 118 must not have adhesive properties, but at the same time it does not impede the rotation of covers 104 with respect to frame 102. Sealant 118 preferably prevents the undesired release of drug 16 through the gap between covers 104 and frame 102. In another embodiment the sealing is accomplished by sealing lips. FIG. 32 shows a partial cross-section “D.” In this embodiment, frame 102 has an elastic protrusion 148, preferably made of rubber, latex, or similar elastic material that has no or low toxicity and is indigestible. Protrusion 148 has grooves on either side. Edges of covers 104 also have elastic protrusions (or lips) 146 made of the same or similar material. Protrusions 146 snugly fit inside the grooves of protrusion 148. This snug fit allows movement required for drug release, yet prevents undesirable release through the gaps between frame 102 and covers 104.

Referring back to FIG. 1, sensors 34a and 34b can be located on the outside surface of capsule 10, but they may also be located inside space 12 leaving the passage for the gastric acid to flow into space 12. In the embodiment where sensors 34 are located inside space 12, all other components located inside space 12 are preferably sealed with a non-digestible material, such as cellulose.

In some embodiments the release of drug 16 may be controlled from the outside by a medical professional or by the patient himself. In these embodiments transmitter 32 or another probe may report on a particular characteristic. This information is transmitted to one or more devices 124, 126, 128 outside the patient's body. These devices are capable of analyzing and outputting information related to the monitored characteristics, the patient, and other information. Once a human determines that the conditions for releasing drug 16 are met, through computer devices 124, 126, 128 provides a signal to capsule 10, which releases the desired amount of drug 16 and according to the desired schedule.

In some embodiments, capsule 10 reports on the successful drug release to one or more devices 124, 126, 128 located outside the patient's body. Capsule 10 may communicate to external devices, such as devices 124, 126, or 128 that the drugs are successfully released and the time when it occurred. Capsule 10 may communicate the event when the processor opened compartments 14. Depending on the embodiment of capsule 10, different sensors may monitor the opening of compartments. This provides assurance that capsule 10 has functioned as desired. Various uses of this information are possible. For example, external computer device 124, 126 or 128 can keep a record of the patient's taking drugs by means of capsules 10 and the change of the characteristics of the condition for which the patient is taking the drug(s) by means of capsule 10. External computer device 124, 126, 128 may have software that would provide further recommendations to the patient, reprogram the remaining capsules 10 for the corrected, more optimal, course of treatment. In some embodiments, the recorded information is transmitted to a storage, where medical professionals can address the departures from the expected course of treatment and communicate with the patient or her external devices, such as devices 124, 126, or 128, or capsules 10.

In some embodiments, capsule 10 reports about component malfunctions. Because capsule 10 is a sophisticated device, malfunction may be possible. The patient or healthcare professional should preferably be aware when capsule 10 malfunctions and drugs contained inside did not get released properly. If drug 16 inside capsule 10 did not get released at all or less of drug 16 was released, capsule 10 reports the malfunction and external computer device 124, 126, or 128 outside the patient's body may alert the patient to take another capsule 10 or to adjust the schedule of taking subsequent capsules 10. The replacement capsule 10 may use the information about the previous capsule 10 malfunction and release more or less of drug 16 or delay the release of drug 16 according to the one or more programs running on processor 22 or according to instruction from the outside device received by receiver 30.

In some embodiments, external computer devices may generate progress and status report and provide other information about health conditions. For example, external computer devices 124, 126, or 128 may present the records of when capsules 10 is activated, for example by swallowing and exposing it to acidic environment of the stomach, the information about the monitored characteristics that the course of treatment is aimed to address, and the timing and dosage of the drug release. The external device may send these report to the medical professional, who in turn can alter the behavior of subsequently taken capsule 10 through external device 124, 126, or 128. For example, if the monitored characteristic exhibits the desired changes in response to treatment, but another characteristic exhibits an undesired change due to the drug's side effect, the medical professional may alter the timing and the dosage of the drug release as she sees best for the patient. External device 124, 126, 128 may alert the patient that the medical professional introduced the change. Naturally, because the patient's health and safety is affected the communication is encrypted and is subject to various verifications to eliminate the possibility of intrusion and tampering with the communicated information.

Microprocessor 22 in capsule 10 analyzes the input that it gets from sensors 34 and/or information that it gets from outside probes or devices through receiver 30. Based on this information, microprocessor 22 decides on the time and the dosage of the drug release. Additionally if capsule 10 contains more than one drug, microprocessor 22 decides on the timing and the dosage of the drug release of each of the drugs it contains. To perform these functions, microprocessor 22 is programmed in a particular way to analyze inputs from sensors 34 and receiver 30 and to cause the opening of the compartments in the particular manner based on the program it is running.

In another embodiment, capsule 10 does not have programs that would enable it to make an independent decision as to the timing and the dosage of the drug 16 release. In this embodiment, when the patient swallows the capsule, she would rely on a medical professional or herself to provide one or more instructions as to the timing and dosage of drug release. This embodiment may be useful in emergency situation when the patient's characteristics are closely monitored by medical professionals.

In some embodiments, information other than what is available through sensors 34 and probes may be considered in determining the timing and dosage of the released drugs. For example, a course of anti-inflammatory drug can span a period of several days. On the first day, the released dosage of the drug is the greatest, diminishing with each passing day. The capsules, which may have the same configuration with respect to the number of compartments and the amount of the anti-inflammatory drug in each compartment, would have the internal calendar. Depending on what date the capsule is activated, it will release more or less of the drug. It should be noted that in this example, all capsules are configured in the same way and only the activation time of a particular capsule is what changes its operation. In another example, the activation time may be a factor, but the capsule would also sense or acquire information about one or more characteristics and would take the information about the characteristic into consideration when releasing the drug. For example, the activation time may be the basis for the maximum amount of the drug being released, while the monitored characteristics informs the capsule to release even less of drug 16.

In some embodiments, it is contemplated that microprocessor 22 is programmed by loading instructions that would dictate the functionality of capsule 10 in memory 28. These instructions do not need to be loaded all at the same time. For example, the manufacturer of capsule 10 may provide instructions for the default behavior of capsule 10. However, a pharmacist can override the default behavior with information that is specific to the patient that is taking capsules 10. For example, capsule 10 may contain the intended drug, however, it should be accompanied by a companion drug. For the sake of the example, one of two drugs may serve the purpose of being the companion drug. The intended drug and both companion drugs are stored in different compartments 14 of capsule 10. The manufacturer preconfigures capsule 10 to release the intended drug together with only the first companion drug. It is known, however, that a significant percentage of the population has an adverse reaction to the first companion drug, which provides for a better effect of the intended drug. If the pharmacist who is filling the prescription for the intended drug has access to the information that a given patient has an adverse reaction to the first companion drug, the pharmacist can override the default behavior of capsule 10, so that the intended drug is released together with only the second companion drug. In some embodiment, the patient's information is uploaded to capsule 10, and microprocessor 22 decides, based on that information, which of the companion drugs to release.

In general, programing of microprocessor 22 may be accomplished in various ways. In one embodiment, the manufacturer, pharmacist, or a medical professional programs the default configuration of capsule 10 that contains a specific drug. In another embodiment, the specifics of the patient are taken in consideration when configuring capsule 10. For example, other drugs that the patient is taking, medical history, allergies, family history, and various other information may is inputted into memory 28. In another embodiment, the patient genetic information is inputted into memory 28. Each type of information (the patient genetic information, non-genetic information, and other information received from sensors and probes) alone or in combination may be useful in deciding on the exact timing and dosage of drug 16 release.

As discussed above, capsules 10 may be programmed with instructions controlling its behavior. In some embodiments, the instructions can be loaded in memory 28 during the manufacture. In many instances at least some instructions can be pre-loaded even before the manufacture of the capsule 10 is complete. When the capsule is filled with drugs by the manufacturer, the manufacturer can load instructions for the particular drug through direct access to microprocessor 22 and memory 28. The manufacturer would not typically have access to the patient information, so capsules are not personalized at this stage. Although embodiments where capsule 10 is personalized for a specific individual based on his or her information at the manufacturing state are also contemplated.

In some embodiments, capsules may be programmed by a pharmacy or another entity that dispenses drugs, such as a hospital. In addition to the generic basic instructions for the operation of capsule 10 related to monitoring one or more characteristics, acquiring certain information from external devices, and controlling the timing and dosage of the released drug, specific patient information can also be input into capsule 10, so it adjusts its behavior accordingly. In some embodiments medical professionals can supply additional information to alter the behavior of capsule 10. This information can come from, for example, observing the patient during treatment.

Programming capsule 10 may be accomplished in a number of ways. The manufacturer may supply instructions and information through direct access to microprocessor 22 and memory 28. FIG. 33 shows apparatus for supplying instructions and information to capsules 10. Container 232 enables the supply of additional information and instructions to capsule 10 inside. Container 232 may enable this in a variety of different ways. For example, container 232 may continuously send an encrypted signal, which puts capsule 10 inside the container in the state, in which its memory can be written into through, for example, wireless communication receiver. In the alternative embodiment, container 232 may generate a magnetic field that puts capsules 10 inside into the state, in which memory 28 can be written into. There may be other modes of enabling the memory writing or a combination of multiple modes may be used. Preferably, such mode or combination of modes ensure against undesirable changes to information stored in memory 28. This can be accomplished by ensuring that the reconfigured capsules are in close proximity to the computer device that writes information into capsules' memory 28, providing an encrypted communication channel, or some by some other combinations of secure communications methods.

Embodiments of capsule 10 enable numerous treatment methods. Some of exemplary methods are set forth below, but the invention is not limited only to these exemplary methods. The sophistication of capsule's 10 programming, determines the variety and complexity of possible applications.

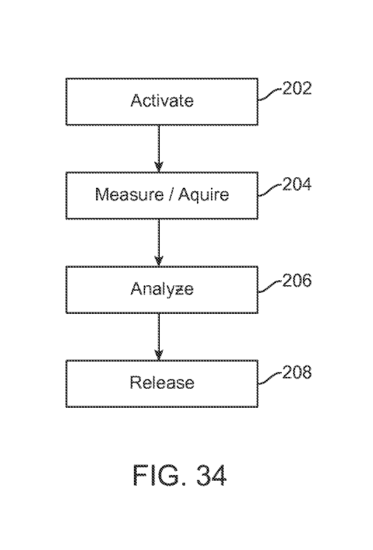

FIG. 34 shows a flow chart of a one-time drug release method that can be accomplished using capsule 10. In step 202, capsule 10 is activated. The activation of the capsule, as discussed above, can be swallowing of the capsule, removing the capsule from its packaging, a computer instruction sent wirelessly to the capsule or any other action or condition capable of activating the capsule. In some embodiments, the capsule may be active at the time when the patient receives it. In step 204 one or more sensors acquire information about one or more monitored characteristics. Alternatively, in step 204, receiver 30 acquires information about one or more monitored characteristics from a probe. In some embodiments, in this step, the medical professional or the patient can supply to memory 28 of capsule 10 additional information such as patient's medical history, genetic information, or other types of information related to the patient. In step 206, microprocessor 22 analyzes the information acquired and determines the dosage of drug 16 needed to be released. In step 208, capsule 10 releases the determined dose of drug 16 by opening one or more compartments 14.

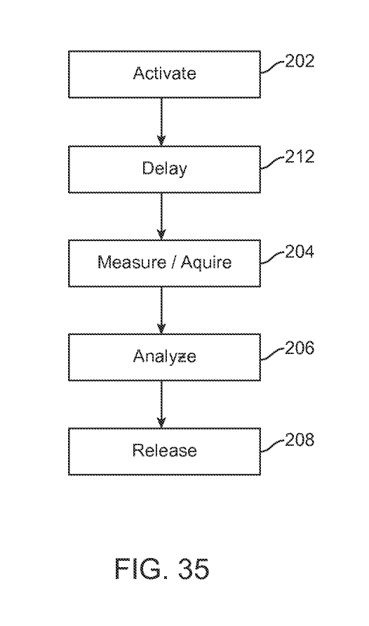

FIG. 35 shows a flow chart of a one-time delayed drug release method that can be accomplished using capsule 10. The method shown in FIG. 35 is similar to the one shown in FIG. 34 except it has an additional step 212 of delay. Specifically, after capsule 10 activation and preferably after the patient has swallowed capsule 10, a delay is introduced before the subsequent steps. The delay may be hard-coded or determined by programs running on microprocessor 22 of capsule 10. The length of the delay can also be communicated by wirelessly communicating information to capsule 10 through receiver 30. After the delay expires, capsule 10 proceeds to step 204 of measuring or acquisition of information about the one or more characteristic of interest, analyzing the information in step 206 and releasing drug 16 in step 208. This delayed method may be useful when the patient needs drug 16 while he is asleep, is undergoing surgery, or in a variety of other circumstances when it is more convenient to swallow a capsule in advance. The delayed release may also be useful when drugs 16 should be released not in the stomach but further downstream in the gastrointestinal tract, for example in the intestines or the colon. It should be noted that delay can be introduced after each step of the drug release method. And multiple delays are contemplated.

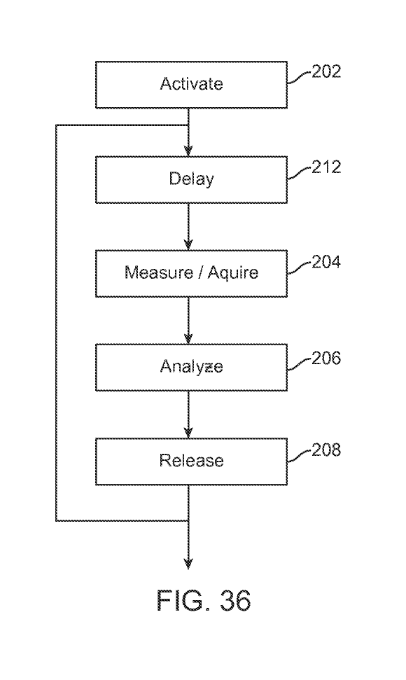

FIG. 36 shows a flowchart for the multiple release. This method is similar to the delayed-release method shown in FIG. 35. In the multiple release method, however, after the initial drug release, microprocessor 22 performs another delay in step 212 and then proceeds to subsequent steps, 204, 206, and 208. This cycle can repeat multiple times. The multiple release method can be useful when drug 16 should be release gradually. The additional step 212 of delay may be introduced at any time of the method. For example, microprocessor 22 may determine, based on the acquired information, the optimal timing of the drug release and introduce a delay according to the determination. Microprocessor 22 may take the time required to open compartments in consideration.

In some circumstances different drugs (such as vitamins, minerals, supplements, etc.) should be taken together or in a certain sequence. Capsule 10 accomplishes this by having different compartments 14 contain different drugs 16. This is useful when the optimal conditions for releasing the first drug require a certain level of the second drug. In this situation, sensors 34 or probe 122 measures the level of the second drug and release the first drug if the levels are acceptable, or alternatively releases sufficient amount of the first drug to bring its levels to the desired ones. As to the steps of the method, microprocessor 22 first releases the first drug and after some predetermined time releases the second drug. This minimizes human error and maximizes the efficiency the drugs. In another embodiment, microprocessor 22 measures or acquires information regarding the level of the first drug. Depending on the measurements, microprocessor 22 releases the desired amount of the first drug, if needed. Microprocessor 22 then, after an optional delay, releases the desired amount of the second drug.

Conversely, some drugs create the risk to health when taken together. Capsule 10 can minimize the risk of such an occurrence. If capsule 10 releases a drug or substance that would have an adverse reaction with another drug or substance, microprocessor 22 tests for the presence of chemicals that would create the adverse reaction. If such a chemical is detected, in some embodiments, microprocessor 22 does not release the drugs. In some embodiments, microprocessor 22 alerts the patient about possible negative drug interaction through external computer device 124, 126, 128. In some embodiments, the patient or the medical professional may decide whether to proceed with the drug release or to override the default behavior.

In some embodiments, it may be desirable for the patient or medical professional to determine the timing and the dosage of the drug release. An external device may obtain relevant information from the probe or from microprocessor 22. Depending on this information and possibly other information collected from the patient's medical history, tests, and observations, the patient or medical professional determines the timing and dosages of one or more drug releases. These information may be communicated to capsule 10 that would carry out the instructions. In this embodiment, capsule 10 contains instruction that accomplish the opening of compartments 14, but the software for analyzing the information and determining when compartments 14 need to open is not necessary.

In some embodiments, capsule 10 may stick to the walls of the esophagus, stomach, or intestine and remain in the patient's body for a period of time that is longer than the usual digestive process. This allows for a prolonged treatment by a single capsule, which reduces the number of capsules required for treatment. Additionally, capsule 10 may collect and analyze data for a longer period of time, which would allow it to make more optimal decisions concerning treatment. Alternatively, capsule 10 may be anchored to a special apparatus or material previously implanted into one or more of the patient's organs.

The foregoing description of the embodiments of the present invention has been presented for purposes of illustration and description. It is not intended to be exhaustive nor to limit the invention to the precise form disclosed. Many modifications and variations will be apparent to those skilled in the art. The embodiments were chosen and described in order to best explain the principles of the invention and its practical applications, thereby enabling others skilled in the art to understand the invention. Various embodiments and modifications that are suited to a particular use are contemplated. It is intended that the scope of the invention be defined by the accompanying claims and their equivalents.

What is claimed is:

1. A drug-containing product comprising:
a. a first partition forming, at least in part, a drug-containing compartment; and
b. a microprocessor capable of causing the drug-containing compartment to open by changing a property of the first partition.

2. The drug-containing product of claim 1, wherein the property of the first partition is physical state, shape, or solubility.

3. The drug-containing product of claim 2 further comprising a sensor capable of monitoring a characteristic, and wherein the processor is capable of determining the timing of the drug-containing compartment opening based on the monitored characteristic.

4. The drug-containing product of claim 2, wherein the property of the first partition is changed through an application of one or more stimuli to the first partition.

5. The drug-containing product of claim 4, wherein the one or more stimuli is one or more of electricity, heat, or light.

6. The drug-containing product of claim 5 further comprising a second partition that, at least in part, forms a control space that contains the microprocessor and the sensor.

7. The drug-containing product of claim 6 further comprising a power source contained in the control space, wherein the power source supplies energy for generation of the one or more stimuli.

8. The drug-containing product of claim 1 further comprising a receiver capable of receiving information and wherein the processor is capable of determining the timing of the drug-containing compartment opening based on the received information.

9. A swallowable capsule comprising:
a. one or more drug-containing compartments; and
b. a control space containing an electronic component, wherein the electronic component is capable of causing the one or more drug-containing compartment to open.

10. The swallowable capsule of claim 9, wherein each of the one or more drug-containing compartments, at least in part, is made of a material capable of changing a property in response to an instruction from the electronic component.

11. The swallowable capsule of claim 10, wherein the material is a shape-memory alloy, shape-memory polymer, piezoelectric material, a transient material, a wax, or a paraffin.

12. The swallowable capsule of claim 9, further comprising a sensor capable of monitoring one or more characteristic.

13. The swallowable capsule of claim 12, wherein the electronic component is capable of analyzing the one or more monitored characteristics and determining the timing of the opening of the one or more drug-containing compartments.

14. The swallowable capsule of claim 9, further comprising a receiver capable of receiving information from an external transmitter.

15. The swallowable capsule of claim 14, wherein the electronic component is capable of analyzing the received information and determining the timing of the opening of the one or more drug-containing compartments.

16. A drug-containing capsule comprising:
a. one or more permanent walls forming, at least in part, a space enclosing control components;
b. one or more modifiable walls forming, at least in part, one or more compartments, each compartment containing a drug; and
c. means for releasing the drugs from the one or more compartments.

17. The drug-containing capsule of claim 16, wherein the means for releasing the drugs causes change to one or more properties of the one or more modifiable walls.

18. The drug-containing capsule of claim 17 further comprising means for determining a timing and a dosage of the drug release.

19. The drug-containing capsule of claim 18 further comprising means for monitoring one or more characteristics.

20. The drug-containing capsule of claim 19, wherein the determining the timing and dosage of the drug release are based on the monitored one or more characteristics.
